(19) United States
(12) Patent Application Publication
(10) Pub. No.: US 2006/0264902 A1
(43) Pub. Date: Nov. 23, 2006

(54) Methods and compositions for treating varicose veins

(76) Inventor: Robert Merchant, Reno, NV (US)

Correspondence Address: KLARQUIST SPARKMAN, LLP, 121 SW SALMON STREET, SUITE 1600, PORTLAND, OR, 97204, US

(21) Appl. No.: 11/414,666

(22) Filed: Apr. 27, 2006

Related U.S. Application Data
(60) Provisional application No. 60/675,525, filed on Apr. 27, 2005.

(51) Int. Cl. A61M 31/00

(52) U.S. Cl. 604/508; 606/159; 424/130.1

(57) ABSTRACT

Certain embodiments of the disclosure provide improved vein closure techniques, such those using radio-frequency radiation. Particular methods include cutting the tip of a catheter. Further examples include locating an incision point by placing a catheter proximate a patient's groin. Yet further examples include sequentially advancing and retracting the catheter during its insertion. Anesthetic may be instilled as the catheter is advanced and retracted. Further embodiments of the disclosure provide improved tumescence fluids, such as those including about 0.1% to about 0.2% lidocaine, about 0.1 to about 1 mg epinephrine per 500 cc of solution, and/or about 10-20 cc of an 8.4% sodium bicarbonate solution per 50 cc of solution. Yet further embodiments provide improved phlebectomy techniques, such as selecting a phlebectomy hook that has the opposite hand of a phlebologist.

### CROSS REFERENCE TO RELATED APPLICATION

This application claims the benefit of, and incorporates by reference, U.S. Provisional Patent Application No. 60/675,525, filed Apr. 27, 2005.

### FIELD

Methods for treating varicose veins are disclosed. Certain aspects provide improved procedures for performing a vein closure, such as techniques using radio-frequency radiation. Compositions useful in such procedures are also disclosed. Further aspects provide improved phlebectomy techniques.

### BACKGROUND

Millions of people suffer from diseases and disorders of the circulatory system. For example, especially as people grow older, they tend to suffer from varicose veins. Varicose veins are abnormally dilated blood vessels, including those having incompetent valves. Incompetent valves remain open and allow blood to pool in the vein, rather than ensuring that blood travels towards the heart

Many people find varicose veins to be cosmetically unattractive. In addition, varicose veins can be painful. Furthermore, untreated, varicose veins can lead to complications such as phlebitis (chronic inflammation of the vein), formation of leg ulcers, and rupture.

Although procedures for treating varicose veins are known, they typically suffer from disadvantages. For example, some treatments require multiple visits to a physician. Some treatments are invasive and require comparatively long recovery times.

### SUMMARY

The present disclosure provides improved methods of treating varicose veins and similar disorders. Certain methods relate to improved methods for closing veins using radio frequency radiation and tumescent anesthesia infiltration. For example, in a particular implementation, a portion of a catheter tip is cut off. The location for an incision is determined by checking the position of the catheter in relation to a patient's groin. The incision is made and a dilator is inserted into the incision. The catheter is inserted, such as into the patient's thigh, and sequentially advanced and retracted as anesthesia fluid is instilled until it is at the desired position. As the catheter approaches the desired position, tumescence fluid is infused into the patient.

When the catheter reaches a muscle, the catheter can be removed, reinserted just under the skin, and then advanced over the muscle. When treatment of the distal portion of the thigh is first completed, a similar method can be carried out on the proximal portion of the patient's thigh up to the groin. In certain implementations, the tumescent fluid catheter can be single or multiple use, is reasonable steerable, and is blunt-tipped, which may aid in avoiding excess trauma and punctures. Pressure, such as by a pump or a blood transfusion cuff, can be used to help force tumescence fluid through the catheter. Further embodiments may lack one or more of the above steps.

In particular implementations, while tumescence fluid is being introduced, the area of interest can be monitored by ultrasound. If the introduction of tumescence fluid is not apparent, such as by an onion-skin effect in the ultrasound, tumescence fluid can be injected by local injection.

Further implementations provide improved vein closure techniques employing one or more of, as described further herein, glidewire marking, foot pedal control of RF energy, methods of treating the portion of the greater saphenous vein near the Hunterian perforator vein, determining when techniques other than RF based techniques may be superior, methods of treating short proximal greater saphenous vein segments, determining the position of the incision point, determining when to apply RF radiation, determining when to avoid sclerotherapy injections, determining when to perform subjunctional ligation, choosing the appropriate size catheter and introducer, methods of using multiple incisions, methods of clearing catheters of debris, and methods of positioning a patient during a vein closure procedure

In particular aspects, the present disclosure provides an improved tumescence fluid. In particular implementations, the tumescence fluid contains an anesthetic, such as about 0.1% to about 0.2% lidocaine, a vasoconstrictor, such as about 0.1 mg to about 1 mg epinephrine per 500 cc solution, and/or a base (or buffering substance), such as about 10 cc to about 20 cc of an 8.4% sodium bicarbonate solution per about 500 cc of tumescence fluid. A particular tumescence fluid has about 0.1% lidocaine, about 0.1 mg epinephrine, and about 16 cc of an 8.4% sodium bicarbonate solution in about 500 cc of tumescence fluid.

Other embodiments provide improved phlebectomy techniques. In one implementation, a method is provided where a phlebologist selects a phlebectomy hook having the opposite hand of the phlebologist. In another implementation, a method of controlling incision width with a dissector is provided. Further implementations increase the spacing between incisions. In particular implementations, a method of phlebectomy of varicose veins is provided where the phlebectomy is performed just below the sapheno-femoral junction.

There are additional features and advantages of the subject matter described herein. They will become apparent as this specification proceeds.

In this regard, it is to be understood that this is a brief summary of varying aspects of the subject matter described herein. The various features described in this section and below for various embodiments may be used in combination or separately. Any particular embodiment need not provide all features noted above, nor solve all problems or address all issues in the prior art noted above.

### BRIEF DESCRIPTION OF THE DRAWINGS

FIG. 1 is a photograph showing various instruments and pieces of equipment which can be used in an improved method of closing varicose veins using radio frequency radiation and tumescent fluid infiltration.

FIG. 2 is a photograph showing additional instruments and pieces of equipment which can be used in an improved method of closing varicose veins using radio frequency radiation and tumescent fluid infiltration.

FIG. 3 is a flowchart of a method of preparing an area for varicose vein treatment using radio frequency radiation and tumescent fluid infiltration.

FIG. 4 is a photograph illustrating how the tip of a catheter can be cut for use in the method of FIG. 3.

FIG. 5 is a photograph illustrating the placement of a catheter in the method of FIG. 3.

FIG. 6 is a photograph of an ultrasound instrument that can be used in the method of FIG. 3.

FIGS. 7-9 are a series of photographs showing how a catheter can be inserted into a patient through a series of advancing and retracting movements, as in the method of FIG. 3.

FIG. 10 is a photograph of an IV bag enclosed within a blood transfusion cuff, which can be used to force tumescence fluid through a catheter in the method of FIG. 3.

FIG. 11 is a photograph of two phlebectomy hooks, each a mirror image (right hand, left hand).

FIG. 12 is a photograph illustrating how a dilator and scalpel can be used to measure an incision.

### DETAILED DESCRIPTION

Unless otherwise explained, all technical and scientific terms used herein have the same meaning as commonly understood by one of ordinary skill in the art to which this disclosure pertains. In the case of conflict, including conflict with references incorporated by reference herein, terms have the meanings provided in the present disclosure.

The singular terms “a,” “an,” and “the” include plural referents unless context clearly indicates otherwise. Similarly, the word “or” is intended to include “and” unless the context clearly indicates otherwise. The term “comprises” means “includes.”

One technique for treating varicose veins is commonly known as the “Closure” technique (CLOSURE is a registered trademark of VNUS Medical Technologies, Inc.). The Closure technique is described in a number of patents, the entire contents of each of which are expressly incorporated by reference herein, including: U.S. Pat. Nos. 6,769,433; 6,752,803; 6,682,526; 6,638,273; 6,613,045; 6,401,719; 6,398,780; 6,322,559; 6,263,248; 6,258,084; 6,237,606; 6,200,312; 6,179,832; 6,165,172, 6,152,899; 6,071,277; 6,036,687; 6,033,398; 6,014,589; 5,810,847, and 5,609,598. Additional information regarding the Closure technique can be found in U.S. Patent Publications 2004 0267258; 2004 0243201; and 2004 0162555; each of which is hereby expressly incorporated by reference in its entirety.

Many methods of treating varicose veins involve completely removing the affected veins from the patient. By contrast, in the Closure technique, the subject vein is closed using heat. During the procedure, a catheter having a radio frequency (RF) generator at its end is inserted into the vein, such as the saphenous vein, and advanced to the uppermost point to be treated. RF radiation is then transmitted from the RF generator.

The RF radiation heats the vein wall (typically to about 85° C. or 185° F.). As the vein wall heats, collagen in the wall of the vein shrinks, thus closing the vein. The catheter is withdrawn through the vein, collapsing the vein behind the catheter tip. Suitable RF generators and catheters are available from VNUS Medical Technologies, Inc. of San Jose, Calif. The position of the catheter can be monitored via ultrasound.

In certain implementations, the tissue in the vicinity of the vein to be closed is treated prior to the application of RF energy. For example, an amount of dilute anesthetic may be introduced into the tissue surrounding the vein to reduce patient discomfort. The treatment may involve introducing a tumescence fluid to the tissue surrounding a vein to be treated. The tumescence fluid swells the tissue surrounding the vein, thus compressing the vein.

The compression of the vein may also force blood from the treatment site. The compression may result in a better fit between the catheter and the vein. In addition to these benefits, use of the tumescence fluid may protect surrounding tissue from being damaged by heat produced by the RF generator. A vasoconstrictor, such as epinephrine, may, in addition to helping to constrict the vein to be treated, limit how quickly the anesthetic is absorbed.

A tumescence fluid may be prepared which has dilute quantities of a base (or buffering substance), vasoconstrictor, and/or an anesthetic, such as a solution including sodium bicarbonate, epinephrine, and lidocaine. A suitable solution may be prepared by taking a 500 cc IV bag of 0.9% saline solution and discarding 50 cc. 50 cc of solution containing 1% lidocaine and epinephrine (epinephrine at a 1:100,000 dilution) is added to the saline solution. 10 cc −20 cc, such as 16 cc, of an 8.4% sodium bicarbonate solution is added to the saline solution. The sodium bicarbonate buffers the acidic nature of the anesthetic and soothes the introduction of the solution into the tissue.

In particular examples, the tumescence fluid contains about 0.1% to about 0.2% lidocaine, preferably 0.1% lidocaine. In further examples, the tumescence fluid contains from about 0.1 mg to about 1 mg of epinephrine per 500 cc of solution, preferably about 0.1 mg. Tumescence solutions having lower concentrations of lidocaine may allow larger volumes of solution to be introduced into a patient.

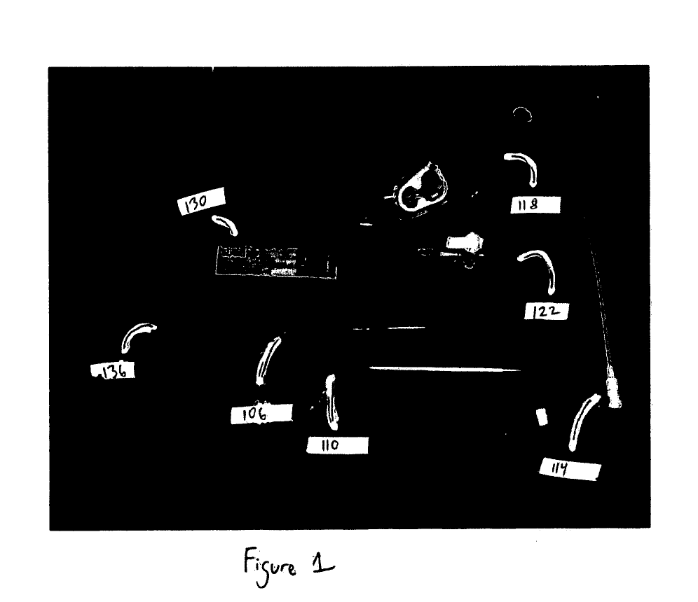

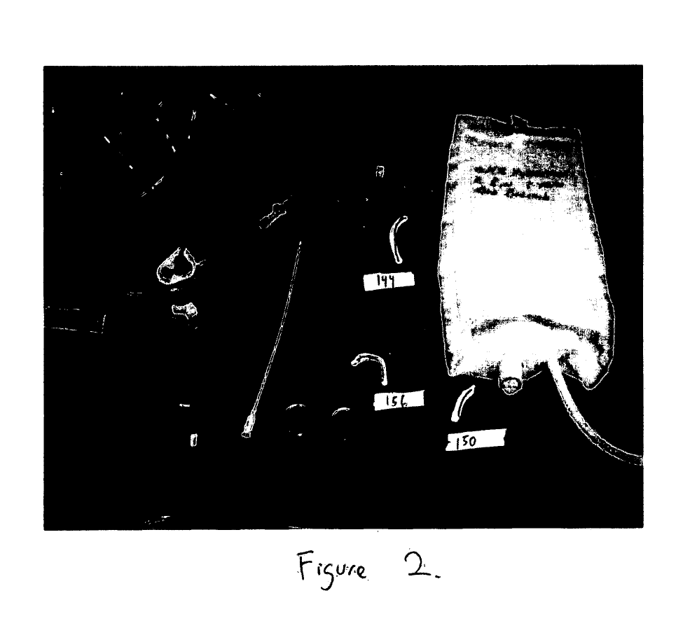

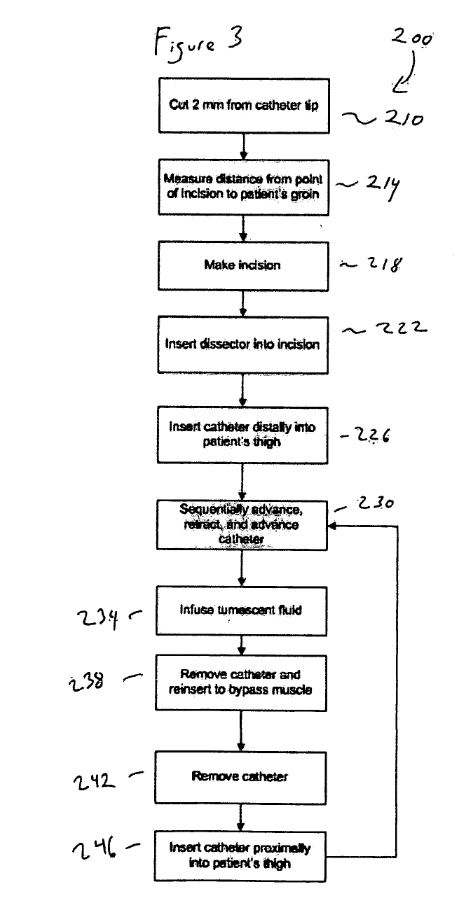

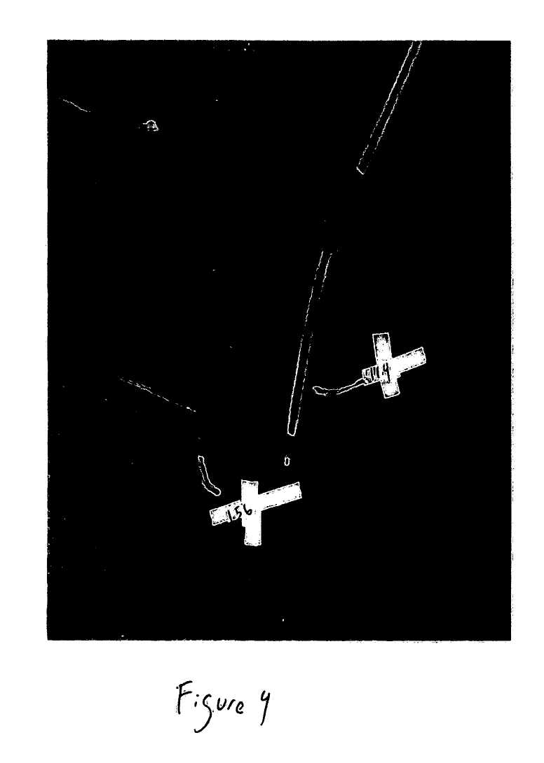

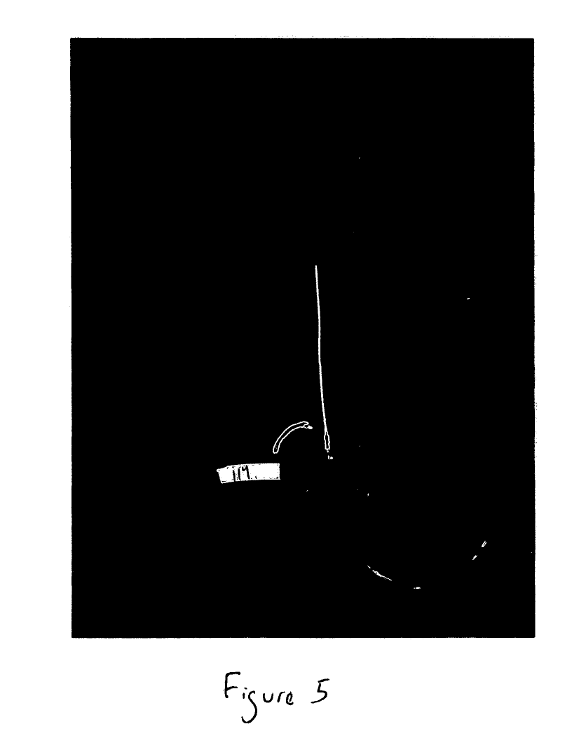

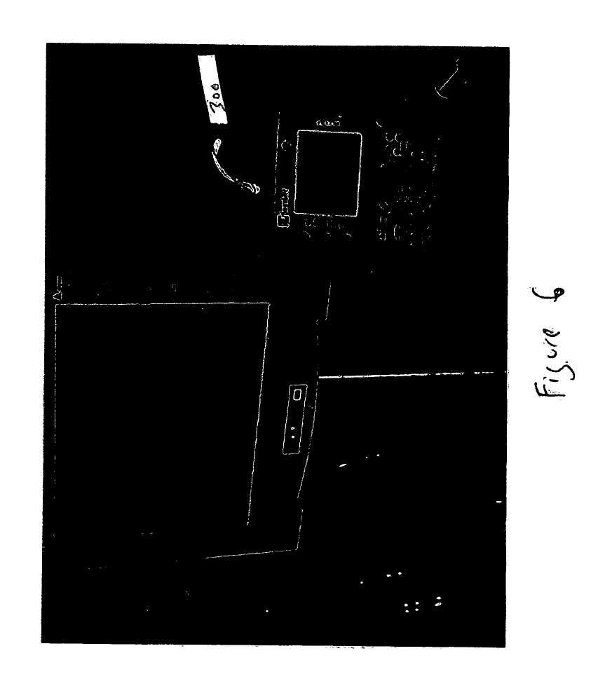

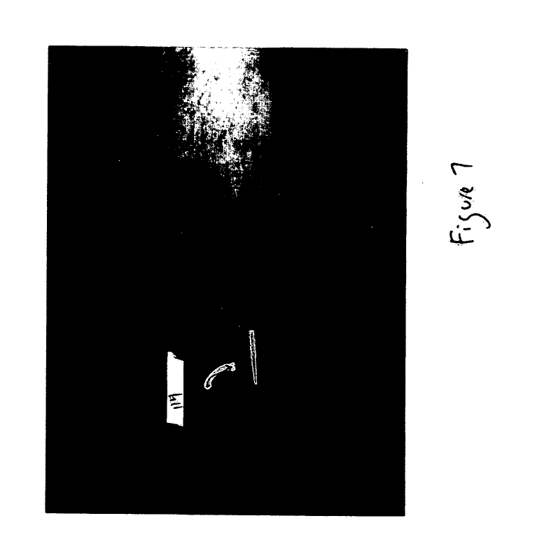

FIG. 1 is a photograph of various instruments and pieces of equipment that can be used in a disclosed improved method for performing a tumescent anesthesia to compliment a Closure procedure. FIG. 1 illustrates instruments and materials that may be used to introduce tumescence fluid into an area to be treated, including a scalpel 106, a dissector 110, a catheter (or dilator) 114, infusion tubing 118, a stopcock 122, a needle 130, and a syringe 136. Additional components are shown in FIG. 2, including an IV bag 144 containing tumescence fluid; a blood transfusion cuff 150, and scissors 156.

The scalpel 106 may be any suitable sharp instrument for making an incision, such as a scalpel with a BP (Bard-Parker) No. 11 blade. The dissector 110 may be any suitable dissector, including a Varady dissector. The catheter 114 may be any suitable catheter, including an 8 French catheter. One suitable catheter is the 405512 catheter available from St. Jude Medical of Minneonka, Minn., having a length of 19 cm and a maximum guidewire outer diameter of 0.038 inches. Suitable catheters 114 will be flexible, so they can be steered when inserted in a patient, yet rigid enough to avoid kinking. The catheters 114 are preferably able to deliver a relatively large amount of tumescence fluid through a single incision, as compared to a “spinal” needles and cannulae, which typically require multiple sticks or incisions.

The infusion tubing 118 may be catalog number ITD available from K.M.I. of Anaheim, Calif. The stopcock 122 may be a four-way stopcock, such as 2C6204, available from Baxter Healthcare Corp of Deerfield, Ill. In certain examples, the stopcock 122 may be adjusted between fully open, fully closed, and manually adjustable positions to help control the rate of infusion. The syringe 136 may be a 10 cc syringe and the needle 130 may be a 25 gauge needle.

FIG. 3 is a flow chart illustrating an improved method 200 for introducing tumescence fluid in preparation for performing a Closure procedure. At step 210, an amount, for example about 2 mm, is cut from the tip of the catheter 114. Step 210 is illustrated in FIG. 4, with the scissors 156 positioned to cut the tip of the catheter 114. Cutting the catheter tip may allow for more rapid infusion of tumescence fluid into the patient, such as by increasing the diameter of the end of the catheter 114, such as by about 0.1 mm to about 0.2 mm. Cutting the catheter tip also blunts and broadens the end of the catheter 114, possibly reducing the chance for damaging veins or other tissue when the catheter 114 is inserted into the patient. Depending on size and characteristics of the catheter 114 used, more or less than 2 mm can be cut from the tip.

At step 214, the catheter 114 is positioned proximate the patient's groin so that the point where an incision should be made into the patient's thigh can be determined. Step 214 is illustrated in FIG. 5. In at least certain embodiments, the patient is in the same position when step 214 is performed as when the Closure procedure is performed.

At step 218, an incision is made in the patient's skin at the point where the catheter 114 will be inserted. In at least one embodiment, the incision is made with a No. 11 blade following infiltration of a small amount of tumescent fluid anesthesia into the skin. The No. 11 blade may be used to create incisions that are comparatively short at the deeper portions of the incision, which may reduce scarring. Although the incision may be any size needed, incisions of about 2 mm are preferred in certain embodiments, as they may produce cosmetically superior results. Although the incisions may be made in any direction, in a preferred embodiment, the incisions are vertical, which may produce cosmetically superior results, such as by minimizing the visibility of any scars.

At step 222, a dissector 110, such as a Varady dissector (FIG. 1), is inserted into the incision to dilate and open up the saphenous fascia. The different layers of the saphenous fascia can be detected through resistance on the dilator 110.

At step 226, the catheter 114 is first inserted distally into the patient's thigh. Ultrasound can be used to help guide the catheter 114 as it is inserted. A suitable ultrasound unit 300 is shown in FIG. 6. Ultrasound can also be used to ensure that the tumescence fluid is entering the proper compartment. For example, the appearance of turbulence in the ultrasound, without a corresponding increase in swelling in the patient, may indicate that there is a problem with the infusion—such as entry of the tumescence fluid into the vein.

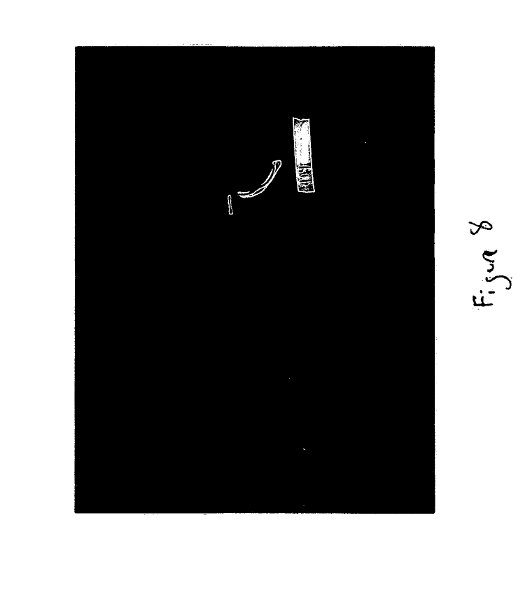

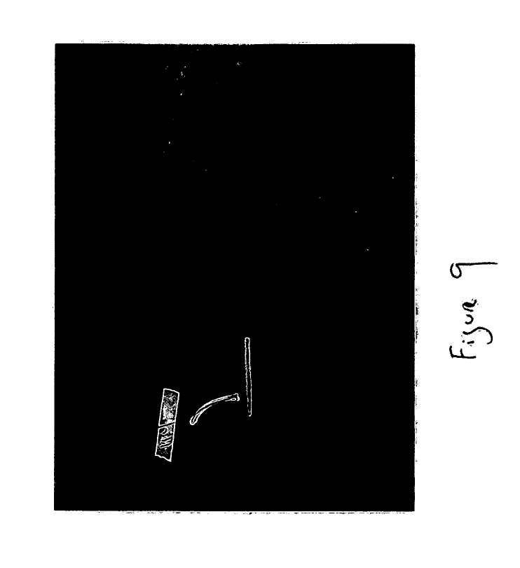

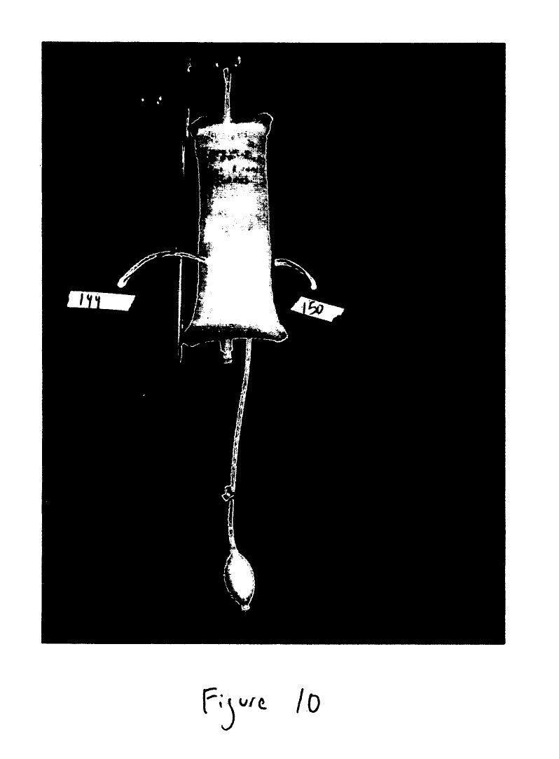

At step 230, the catheter 114 is repeatedly advanced, such as about 1 cm at a time, and then retracted, and advanced again. In this way, the catheter 114 is gradually advanced into the patient's tissue. The steps of advancing, retracting, and advancing the catheter 114 are illustrated in FIGS. 7-9. This helps produce a smooth and painless introduction of the tumescent anesthetic solution.

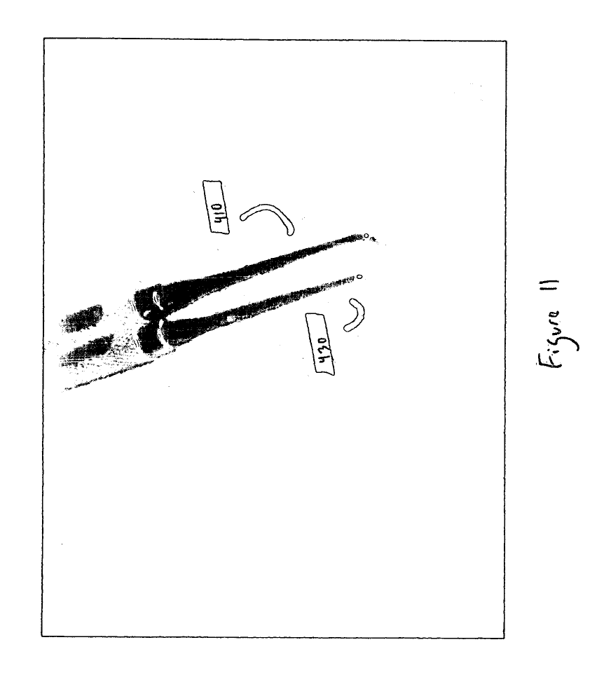

At step 234, tumescence fluid is infused into the patient through the catheter 114. For example, as shown in FIG. 10, when the IV bag 144 is in blood transfusion cuff 150, the blood transfusion cuff 150 can be inflated to a desired pressure, such as about 150 mm Hg. Of course, other methods can be used to force tumescent fluid through the catheter 114, such as a mechanical pump (e.g., a Klein pump). The fluid flow can also be regulated using the stop cock 122.

At step 122, when the catheter 114 encounters a muscle, such as the sartorius muscle, the catheter 114 is withdrawn to the introduction incision. The catheter 114 is then redirected just below the patient's skin, and advanced over the muscle. This method of inserting the catheter 114 can result in less trauma to the patient and decrease healing time and discomfort.

At step 242, the catheter 114 is removed from the patient.

At step 246, the catheter 114 is inserted proximally into the patient's thigh. Tumescence fluid is introduced in a manner analogous to steps 226-242.

While the tumescence fluid is being introduced into the area to be treated, it can be visualized by ultrasound where it creates an “onion-skin” appearance surrounding cross-cut sections of the vein. If this effect is not obvious at a particular area, additional tumescence fluid can be injected at the area by local injection using the syringe 136 and the needle 130.

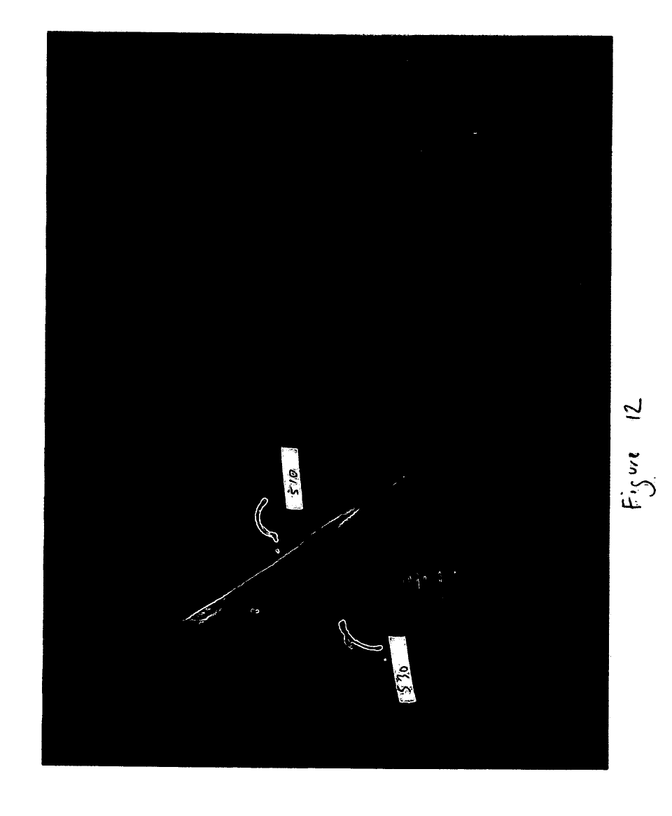

Additional aspects provide improved phlebectomy techniques. For example, as shown in FIG. 11, phlebectomy hooks are typically available with hooks that curve either counterclockwise 410 or clockwise 430. Typically, a phlebologist selects a particular hook depending on whether they are left handed or right handed. However, the use of a single hook can result in awkward hand positions and movements being needed to remove veins in certain position. It has been found that, in these situations, greater control and more facile vein removal can be achieved by switching to the opposite handed hook.

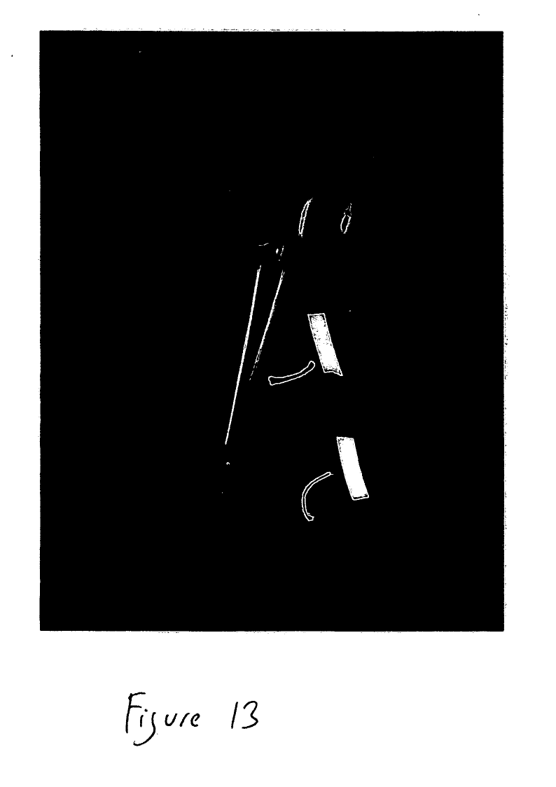

Phlebologists typically control the width of an incision by positing a cutting instrument with a clamp. However, as illustrated in FIG. 12, greater control may be achieved by using a dissector 510 proximate a cutting instrument 530, such as a scalpel, thereby selectively dilating the incision once a shallow incision has been made.

Prior phlebectomy techniques typically use incisions spaced 1 inch apart. However, it has been found that the spacing between incisions can be increased by strategically locating the incision. For example, the incision may be located at a point of common vein branching. In certain embodiments, the patient is placed in a supine or prone position. The veins are located with the use of a Vein Lite (available from, and trademark of, Translite of Sugar Land, Tex.), or similar transilluminating device, and the location marked on the patient's skin. The physician can then choose the optimum position to make incisions.

Using this technique, the spacing between incisions can be increased to between about 3 inches and about 4 inches. Fewer incisions may result in a faster procedure, shorter recovery time, reduced number or frequency of complications, and improved cosmetic results.

Typically, during phlebectomy of the varicose veins, the sapheno-femoral junction, or the anterior or posterior saphenous branches, the physician ties all of the veins off and performs a high ligation of the sapheno-femoral junction. However, satisfactory results can be achieved by simply performing a phlebectomy just below the junction, leaving other normal tributaries intact. This procedure may result in greater preservation of healthy vein tissue, immediately and long-term. Contrary to the typical understanding, over time, the tributaries near the sapheno-femoral junction remain competent. This is also true for the sapheno-popliteal junction behind the knee. This reduces the development of neovascularization, or reattachment of varicosities to the deep vein, which can contribute to early reoccurrence of venous hypertension and, this, new varicose veins.

A number of additional improvements to the Closure procedure are summarized below:
The glidewire can be marked to note the length of the selected catheter at the hub attachment for infusion. The catheter can then be inserted over the glidewire to the point where the mark on the glidewire coincides with the catheter length (typically 45, 60, or 100 cm).
During the Closure procedure, a foot pedal can be used to control the application of RF energy during the procedure. The physician's foot is preferably placed near the foot pedal for about the first 10-15 seconds after the initiation of RF radiation. The position of the foot pedal is preferably noted at all times by the physician so that the RF radiation can be turned off if the patient experiences any heating sensation. If a foot pedal is not used, a nurse or assistant should remain near the power switch during treatment. If the procedure needs to be stopped, and the foot pedal cannot be located, the catheter can be removed in one motion.
When the superficial branch of the greater saphenous vein arising near the Hunterian perforator vein is to be treated, it may be desirable to treat only that portion of the proximal greater saphenous vein which lies relatively deep in the thigh with RF radiation and to perform an ambulatory phlebectomy of the portion that is superficial. Such a method of treatment can increase patient comfort and be cosmetically superior. The vein should be ligated to reduce the likelihood of hemorrhage, or bleeding, during the post operative period.
Cosmetically superior results may be obtained using a technique other than the Closure technique, e.g. ambulatory phlebectomy, not stripping, to treat veins which are easily seen beneath the skin, particularly in patients with low body fat or low body mass index.
When treating short proximal greater saphenous vein segments, such as those from about 10 to about 15 cm, it may be beneficial to ligate the proximal segment at the site of introduction. Early post-operative ambulation by the patient, if accompanied with activities such as vomiting, coughing, stooping, bending over, might result in inadvertent opening of the proximal segment and hemorrhage from the vein entry site.
When performing a closure of the small, or lesser, saphenous vein, it may be useful to consider the diameter of the vessel that will be treated. The entry site should allow for at least 5-6 cm of Closure from the original heat spot. In addition, it may be beneficial to begin applying heat where the RF catheter first becomes parallel with the overlying skin as seen by ultrasound, usually where the gastrocnemius muscle begins to curve downward into the popliteal space, typically about 3 cm from the sapheno-popliteal junction. The neurovascular structures have separated from the small saphenous vein proximal to this point and patients should not experience pain at the onset of RF heating.
Sclerotherapy injections should be avoided with RF procedures because the solution may migrate through the treated saphenous vein, which is not completely occlusive immediately following the Closure procedure, and this may affect the endothelium at the sapheno-femoral junction and result in clot extension into the deep vein.
Avoid proximally locating the RF source within about 1 cm to about 2 cm of the sapheno-femoral junction because heat from the greater saphenous vein wall might cause unintentional injury at the sapheno-femoral junction that might result in a greater risk of clot extension into the common femoral vein.
Prior to activating the RF source, check the placement of the catheter using ultrasound. The catheter is preferably located about 1 to about 2 cm from the junction. Tumescence fluid should surround the junction and the entire segment of the vein to be treated. The tumescence fluid may act on the vein segment to stretch it over the catheter, sometimes causing it to move closer to the sapheno-femoral junction.
When the proximal greater saphenous vein is widening or wide (such as greater than about 12 mm) at the sapheno-femoral junction, subjunctional ligation may be preferred to lessen the possibility of clot extension into the common femoral vein, as it becomes a controlled endpoint for vein occlusion.
If a 6 French catheter is being used for the Closure procedure and repeated shut-downs occur after cleaning the catheter, the possibility of acute thrombosis of the vein segment should be considered. This can be confirmed with ultrasound. If thrombus is present, it may be desirable to switch to an 8 French catheter, including an 8 French introducer. In certain examples, the 8 French catheter is used with a 0.038 inch guidewire.
Multiple incisions may be better than trying to perform an entire closure procedure from a single incision. For example, when treating bifid great saphenous vein segments, consider treating each separately with separate venipunctures and tumescent anesthesia. In such cases, it may be desirable to treat the distal segment first so as not to distort the proximal sapheno-femoral junction by tumescence fluid.
If a clot develops in the greater saphenous vein while the Closure procedure is being performed, consider switching to an 8 French catheter and adding a blood transfusion pressure cuff to the catheter infusion bag to keep fluid flowing.
If one uses an RFS-Flex catheter or similar device, degraded blood product debris can lead to early and frequent electrical shutdown of the RF generator. To avoid or lessen this problem, a careful continuous “to and fro” movement of the catheter, approximately 5 mm at a time can provide a “self-cleaning” effect.
Use a 19 cm, 8 French dilator to deliver tumescence fluid under ultrasound guidance.
Until the Closure procedure is well underway, do not change the patient's leg position once the position of the RF source has been established by ultrasound. Otherwise, the catheter may advance up the vein, possibly into the deep vein.

It is to be understood that the above discussion provides a detailed description of various embodiments. The above descriptions will enable those skilled in the art to make many departures from the particular examples described above to provide apparatuses constructed in accordance with the present disclosure. The embodiments are illustrative, and not intended to limit the scope of the present disclosure. The scope of the present disclosure is rather to be determined by the scope of the claims as issued and equivalents thereto.

We claim:

1. A varicose vein treatment method comprising:
removing the tip of a catheter;
locating an incision point on a patent's thigh by checking the position of the catheter in relation to the patient's groin;
making an incision in the patient at the incision point;
inserting a dilator into the incision;
inserting a catheter into the incision;
sequentially advancing and retracting the catheter in the patient's thigh;
as the catheter is sequentially advanced and retracted, instilling anesthesia fluid through the catheter; and
as the catheter approaches a desired position, infusing tumescence fluid into the patient through the catheter.

2. A tumescence fluid comprising:
(A) about 450 cc of 0.9% saline solution;
(B) about 50 cc of a 1% lidocaine and epinephrine solution, comprising epinephrine at a 1:100,000 dilution; and
(C) about 10 to about 20 cc of an 8.4% sodium bicarbonate solution.

3. A phlebectomy method comprising selecting a phlebectomy hook having the opposite hand of a phlebologist.
